(19) United States
(12) Patent Application Publication
(10) Pub. No.: US 2011/0071117 A1
(43) Pub. Date: Mar. 24, 2011

(54) COMBINATIONS OF ANTIOXIDANTS AND ANTI-INFLAMMATORY DRUGS IN THE TREATMENT OF INFLAMMATORY BOWEL DISEASE

(75) Inventor: Richard F. Harty, Oklahoma City, OK (US)

(73) Assignee: The Board of Regents of the University of Oklahoma, Norman, OK (US)

(21) Appl. No.: 12/992,288

(22) PCT Filed: May 14, 2009
(86) PCT No.: PCT/US09/43892

§ 371 Date: Nov. 12, 2010

Related U.S. Application Data
(60) Provisional application No. 61/053,473, filed on May 15, 2008.

(51) Int. Cl. A61K 31/606; A61P 39/06; A61P 29/00

(52) U.S. Cl. 514/161; 514/166; 514/162

(57) ABSTRACT

Disclosed herein are compositions comprising 5-aminosalicylic acid (5-ASA), or a pharmaceutically acceptable salt thereof and an antioxidant, or a pharmaceutically acceptable salt thereof. Also disclosed herein are methods of treating an inflammatory bowel disease in a subject, reducing cytokine gene expression in colonic tissue of a subject, and reducing myeloperoxide activity in colonic tissue of a subject, comprising administering to the subject a therapeutically effective amount of a composition comprising 5-ASA, or a pharmaceutically acceptable salt thereof, and an antioxidant, or a pharmaceutically acceptable salt thereof.

### RELATED APPLICATIONS

The present application claims priority to the U.S. Provisional Application Ser. No. 61/053,473, filed on May 15, 2008 by Richard F. Harty and entitled “COMBINATIONS OF ANTIOXIDANTS AND ANTI-INFLAMMATORY DRUGS IN THE OF TREATMENT INFLAMMATORY BOWEL DISEASE,” the entire disclosure of which, including the drawings, is incorporated by reference herein.

### FIELD OF THE INVENTION

The present invention is in the field of combination drug therapy, and particularly in the field of treatment of inflammatory bowel disease using anti-inflammatory drugs and antioxidants.

### BACKGROUND OF THE INVENTION

Reactive oxygen species (ROS) have been associated with the initiation or aggravation of a number of illnesses including inflammatory bowel disease (IBD). Excessive generation of ROS can induce biochemical alterations in macromolecules such as lipids, proteins, and DNA and, thereby, induce cellular injury or death in the inflammatory process. Alternatively, the duality of ROS effects is reflected by the ability of these oxygen and nitrogen radicals to positively affect intracellular signaling pathways and metabolism. In this latter instance, ROS are formed as byproducts of oxygen metabolism under physiological or non-stress conditions and their levels are regulated by both non-enzymatic and enzymatic antioxidants. Oxidative stress encountered in inflammatory states and in ischemia and reperfusion injury represents a disequilibrium or imbalance between the generation of reactive oxygen intermediates and the removal of these species by antioxidant systems. Exposure of cells to oxygen radicals can activate or trigger surface receptors and cellular pathways that can promote either cell survival or death. The fate of the cell experiencing oxidative stress depends, in part, on the cell type and the duration and magnitude of exposure and the dominance of opposing cellular signals.

Therapeutic approaches to clinical and experimental forms of IBD have, heretofore, been broad based and non-selective with aims to suppress the inflammatory process and immune over reactivity. These strategies have reflected our incomplete and fragmented understanding of the pathogenesis of ulcerative colitis and Crohn's disease.

More recent investigations have expanded and emphasized the apparent linkage between genetics, immune cell function and luminal bacteria recognition in these chronic inflammatory diseases. Mesalamine (5 aminosalicylic acid; 5-ASA) has been used in various oral and enema/suppository formulations for mild to moderate ulcerative colitis and also in Crohn's disease. 5-ASA exerts its anti-inflammatory effects through several pharmacological actions. These include its antioxidant properties and the ability to inhibit prostaglandin synthesis and reduce nuclear factor-κB (NF-κB) activation. The role of oxidative stress in experimental models of colitis has been investigated directly and indirectly in studies employing topical and systemic antioxidant agents. Improvement in measures of inflammation by these agents has been ascribed to reduction in ROS, inhibition of nitric oxide synthase (NOS) and inhibition of the activation of NF-κB. These studies indicate the presence of similarities in certain aspects of the pharmacological actions of mesalamine and antioxidants.

Therapeutic interventions in experimental colitis, for example those described in U.S. Pat. No. 7,417,037, incorporated by reference herein in its entirety, have examined the ability of the antioxidant N-acetylcysteine (NAC) in combination with mesalamine to reduce inflammation and promote healing in trinitrobenzene sulfonic acid (TNBS) induced colitis in rats. The rationale for this therapeutic approach was to take advantage of the shared and distinctive actions of each agent in a combined formulation. This pharmacological approach provided the ability to modulate a number of components of the inflammatory cascade in an immune-mediated model of colitis. Experimental results indicated that the combination of N-acetylcysteine plus 5-ASA when administered rectally to rats with TNBS colitis was superior to either agent alone in promoting mucosal healing and reducing inflammation. Furthermore, combination therapy resulted in significant reductions in colonic myeloperoxidase activity and proinflammatory cytokine gene expression. Additional studies have demonstrated that NAC plus 5-ASA exerted its therapeutic benefit, in part, by inhibiting the proinflammatory actions of prostaglandin E₂ (PGE₂) and offsetting the deleterious effects of oxidative and nitrosative stress induced by TNBS colitis.

### SUMMARY OF THE INVENTION

Disclosed herein are compositions comprising 5-aminosalicylic acid (5-ASA), or a pharmaceutically acceptable salt thereof and an antioxidant, or a pharmaceutically acceptable salt thereof.

Also disclosed herein are methods of treating an inflammatory bowel disease in a subject, reducing cytokine gene expression in colonic tissue of a subject, and reducing myeloperoxide activity in colonic tissue of a subject, comprising administering to the subject a therapeutically-effective amount of a composition comprising 5-ASA, or a pharmaceutically-acceptable salt thereof, and an antioxidant, or a pharmaceutically acceptable salt thereof.

### BRIEF DESCRIPTION OF THE DRAWINGS

FIG. 1 is a graph showing the macroscopic injury scores from the distal colon of animals 11 days after TNBS and 8 days of treatment. Animals treated with saline alone served as control group. Treatment protocols were initiated 3 days after induction of TNBS colitis and comprised daily rectal dosing with saline, 5-ASA (100 mg/kg), Vitamin C (100 mg/kg)+5-ASA, PBN (30 mg/kg)+5-ASA and Vitamin E (50 mg/kg)+5-ASA. *P<0.01 vs TNBS alone. N/S denotes non significant.

FIG. 2 is a graph showing the global microscopic injury scores from distal colon of animals 11 days after TNBS and 8 days of treatment. Individual treatments were initiated 3 days after induction of TNBS colitis and included daily administration of one of the following: 5-ASA (100 mg/kg), Vitamin C (100 mg/kg)+5-ASA, PBN (30 mg/kg)+5-ASA and Vitamin E (50 mg/kg)+5-ASA. *P<0.01, ±P<0.0001 vs TNBS alone. **P<0.01, †P<0.004 vs 5-ASA alone. N/S denotes not significant.

FIG. 3 is a graph showing the subset analysis of individual indices of TNBS-induced microscopic colitis results with 5-ASA alone are compared to responses with each antioxidant plus 5-ASA. *P<0.04 vs 5-ASA only. **P<0.001 vs 5-ASA only. † P<0.007 vs 5-ASA only.

FIG. 4 is a graph showing the myeloperoxidase activity in distal colon tissues from animals 11 days after TNBS and 8 days of treatment with: saline, 5-ASA alone, 5-ASA+Vitamin C, 5-ASA+PBN and 5-ASA+Vitamin E. *P<0.05, **P<0.002 vs TNBS alone. †P<0.04 vs 5-ASA alone. N/S denotes non-significant.

### DETAILED DESCRIPTION OF THE INVENTION

Aspects of the present disclosure relate to combinations of antioxidants other than N-acetylcysteine and 5-ASA and their ability to affect the ulcerative and inflammatory responses to TNBS and promote mucosal repair. In some embodiments, the antioxidant agents are vitamin C (ascorbic acid), vitamin E (α-tocopherol) and phenyl butylnitrone (PBN).

Thus, in one aspect, disclosed herein is a composition comprising 5-aminosalicylic acid (5-ASA), or a pharmaceutically acceptable salt thereof and an antioxidant, or a pharmaceutically acceptable salt thereof.

The term “pharmaceutically acceptable salt” refers to a formulation of a compound that does not abrogate the biological activity and properties of the compound. Pharmaceutical salts can be obtained by reacting a compound with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

In some embodiments, the antioxidant is selected from the group consisting of vitamin C (ascorbic acid), vitamin E (α-tocopherol), and phenyl butylnitrone. In other embodiments, the antioxidant is a mixture of two or more antioxidants.

In some embodiments, the composition further comprises a pharmaceutically acceptable carrier or excipient.

In certain embodiments, the 5-ASA, or a pharmaceutically acceptable salt thereof, and the antioxidant, or a pharmaceutically acceptable salt thereof, are together disposed in a liquid formulation. In some embodiments, the liquid is an aqueous solution, whereas in other embodiments, the liquid is an emulsion. In other embodiments, the 5-ASA, or a pharmaceutically acceptable salt thereof, and the antioxidant, or a pharmaceutically acceptable salt thereof, are together disposed in a solid formulation.

In another aspect, disclosed herein is a method of treating an inflammatory bowel disease in a subject, comprising administering to the subject a therapeutically-effective amount of a composition comprising 5-ASA, or a pharmaceutically-acceptable salt thereof, and an antioxidant, or a pharmaceutically acceptable salt thereof.

In another aspect, disclosed herein is a method of reducing cytokine gene expression in colonic tissue of a subject comprising administering to the subject a therapeutically-effective amount of a composition comprising 5-ASA, or a pharmaceutically-acceptable salt thereof, and an antioxidant, or a pharmaceutically acceptable salt thereof. In some embodiments, the cytokine is selected from the group consisting of IL 1a, IL 1b, IL-4, IL-6, and TNFα.

In another aspect, disclosed herein is a method of reducing myeloperoxide activity in colonic tissue of a subject comprising administering to the subject a therapeutically-effective amount of a composition comprising 5-ASA, or a pharmaceutically-acceptable salt thereof, and an antioxidant, or a pharmaceutically acceptable salt thereof.

In some embodiments of the methods disclosed herein, the composition is administered to the subject by rectal delivery or oral delivery to a colon or small intestine of the subject. Those of skill in the art recognize that by administering a composition, for example a liquid composition, rectally, the composition can move upstream from the colon and enter the small intestine, for example the ileum or jejunum. In some embodiments, a rectally delivered composition is only administered to the colon, whereas in other embodiments, the rectally delivered composition reaches further upstream and enters the small intestine through the ileo-cecal valve.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes; and, in particular, humans. Preferably, the subject is a human.

In some embodiments of the methods disclosed herein, the 5-ASA, or a pharmaceutically-acceptable salt thereof, and the antioxidant, or a pharmaceutically-acceptable salt thereof, are together disposed in a pharmaceutically acceptable carrier.

In certain embodiments of the methods disclosed herein, the 5-ASA, or a pharmaceutically-acceptable salt thereof, and the antioxidant, or a pharmaceutically-acceptable salt thereof, are administered separately. In these embodiments, the two compounds are not formulated together in a single dosage form. Instead, the subject, i.e., the subject, takes one dosage form containing one of the 5-ASA, or a pharmaceutically-acceptable salt thereof, or the antioxidant, or a pharmaceutically-acceptable salt thereof, and then takes another dosage form containing the other of the 5-ASA, or a pharmaceutically-acceptable salt thereof, or the antioxidant, or a pharmaceutically-acceptable salt thereof. In some of these embodiments, the subject takes the two dosage forms simultaneously, i.e., the subject takes the second dosage form within minutes, preferably less than 10 minutes, from the taking of the first dosage form. In other embodiments, the subject takes the two dosage forms nearly simultaneously, i.e., the subject takes the second dosage form within one hour of taking the first dosage form. In other embodiments, the time interval between the taking the two dosage forms is greater than one hour.

In some embodiments, the methods described herein cause macroscopic injury to colonic mucosa of the subject to be reduced. In other embodiments, the methods described herein cause microscopic injury to colonic mucosa of the subject to be reduced. In further embodiments, the methods described herein cause inflammation in colonic mucosa of the subject to be reduced.

In some embodiments of the methods disclosed herein, the antioxidant is selected from the group consisting of vitamin C (ascorbic acid), vitamin E (α-tocopherol), and phenyl butylnitrone.

The methods and results disclosed herein show that α-tocopherol, in combination with 5-ASA, caused significant and synergistic reductions in global and individual indices of microscopic colitis and colonic MPO activity. Vitamin C or ascorbic acid in combination with 5-ASA also decreased significantly the cumulative microscopic injury score and the subset analysis for ulceration. Furthermore, the level of MPO activity observed with vitamin C plus 5-ASA was significantly less than that recorded for TNBS. α-Tocopherol or vitamin C plus 5-ASA acted topically in rats afflicted with immune-mediated colitis to promote reduction in inflammation and mucosal healing to a degree that was significantly greater than mesalamine alone.

The results presented with regard to vitamin E demonstrate similarities to previous studies with NAC plus 5-ASA in the terms of the magnitudes of reduction in cumulative and subset parameters of microscopic colitis and MPO activity. Specifically, NAC plus 5-ASA and α-tocopherol plus 5-ASA each reduced the global TNBS colitis injury score to levels that were significantly less than those recorded for 5-ASA alone. Furthermore, subset analysis of individual indices of colitis demonstrated that both NAC plus 5-ASA and vitamin E plus 5-ASA reduced significantly each parameter of microscopic colitis. In addition, MPO activity as a biomarker of polymorphonuclear (PMN) leukocyte infiltration in the colon was reduced by greater than eighty percent with each combination therapy. These findings paralleled the dramatic reductions in PMN numbers in the lamina propria assessed quantitatively by microscopy. It is also worth noting that the antioxidant vitamin C when added to 5-ASA enema preparation also caused significant enhancement in the anti-inflammatory actions of mesalamine. Taken together, the present and previous studies from our laboratory suggest that the addition of antioxidant agents to topically applied mesalamine can significantly augment the anti-inflammatory and healing processes in the treatment of colitis in this experimental model.

It is likely that the combined effects of vitamin E and 5-ASA result in reduction in the elaboration of proinflammatory cytokines evoked by TNBS immune-mediated colitis. An indirect indicator of immune mediator down regulation is the profound decrease in mononuclear cell infiltrate observed in the distal colon with vitamin E plus 5-ASA treatment. In similarly designed studies, NAC plus 5-ASA inhibited cytokine gene expression elicited by TNBS in the rat colon. Among the cytokines examined (IL-1A, IL-1B, 1L-04, IL-6 and TNFα) gene expression was inhibited significantly by NAC plus 5-ASA, whereas the results with either NAC or 5-ASA as single agents did not achieve this degree of statistical distinction. Furthermore, the TNBS-associated marked increases in colonic levels of PGE₂ and NO were counteracted by treatment with NAC plus 5-ASA. These findings suggest that combination therapy with NAC plus 5-ASA may disrupt a critical link between the proinflammatory effects of PGE₂ and oxidative stress in this model of inflammatory bowel disease.

Investigational experience with α-tocopherol as a treatment in experimental models of IBD is limited. On the other hand, there exists an extensive bank of literature on the biology of vitamin E in atherogenesis and cancer chemoprevention. α-Tocopherol, as a member of the vitamin E family, possesses both antioxidant and non-antioxidant properties. The role of α-tocopherol as an antioxidant has been studied extensively in relationship to lipid peroxidation, LDL metabolism and atherosclerosis. The chemistry is complex and favorable experimental results have not translated into demonstrated utility of dietary supplementation with vitamin E to positively affect the inflammatory process of atherosclerosis. Emerging interest in tocopherols and other antioxidant/anti-inflammatory agents in chemoprevention of cancer, including colon cancer, is an area of active inquiry both in the laboratory and clinic. A consequence of these and other studies is an expanded appreciation of the actions of tocopherols in different cell systems. As an essential lipid soluble vitamin of plant origin vitamin E is taken up by the intestine in a bile acid-dependent manner and then transported in chylomicrons to the liver. Hepatocyte uptake of α-tocopherol is facilitated by a tocopherol transfer protein (TTP) and, thereby, incorporated into nascent VLDL. Cholesterol biosynthesis within cultured hepatocytes has been reported to be inhibited by vitamin E at the transcriptional level and to be independent of its antioxidant action. The oxidation of LDL has been recognized as a key factor in the genesis of inflammatory atherosclerotic lesion in arteries. The ability of α-tocopherol to interrupt the autoxidation of LDL fatty acid peroxyl radicals in vitro may not extend to in vivo conditions because of the paradoxical role of α-tocopherol to act as pro-oxidant of LDL. The pro-oxidant potential of tocopherols and other antioxidants must be considered in their spectrum of action and, in particular, in the area of cancer prevention. Despite this caveat, tocopherols and other antioxidants have been shown to modulate immune and inflammatory cell function and intracellular signaling systems in ways that may positively influence disease progression.

The administration of a variety of antioxidants by oral, parenteral and intracolonic routes have, in general, been shown to reduce colonic inflammation induced by agents such as DSS and TNBS. The rationale for and impact of these studies is to underscore the role of oxidative and nitrosative stress in the pathogenesis of experimental and clinical inflammatory bowel disease. Conceptually, it is reasonable to envision that the elaborations of reactive oxygen/nitrogen species and soluble mediators of inflammation occur following the initiation of a dysregulated immune response to an inciting event(s) in IBD. Therapeutic abrogation of oxidative stress and reduction in proinflammatory factors that results in amelioration of experimental colitis supports, on the one hand, their prominent place in the inflammatory cascade and, on the other, suggests that restoration of the levels of these substances to or toward normal can have a homeostatic effect on activated immune cells which perpetuate the inflammatory process. One unique aspect of the present disclosure is that each of the agents with antioxidant properties was administered topically with mesalamine by enema to the distal colon of rats with acute TNBS colitis. Although there have been no reported comparable studies on the topical effects of vitamin E in experimental colitis, the effects of a water-soluble vitamin E derivative, administered by intraperitoneal injection for 7 days, on TNBS colitis in rats has been examined. It has been observed that this therapy significantly reduced colonic inflammation, MPO activity, cytokine production and lipid peroxidation. In the present study α-tocopherol was solubilized with DMSO and co-administered with mesalamine by enema for 8 days beginning 3 days after induction of TNBS colitis. It is of note that the overall colitis damage score reported by Isozaki et al (“Effect of a novel water-soluble vitamin E derivative as a cure for TNBS-induced colitis in rats”, Int J Mol Med 17: 497502, 2006) was reduced by approximately 60% with ip administration of the water-soluble tocopherol derivative while the present study observed 82% decrease in global microscopic colitis with rectally dosed α-tocopherol plus mesalamine. Furthermore, MPO activity in the Isozaki et al study was maximally reduced by 43% while topical treatment with α-tocopherol plus mesalamine, in the present study, decreased colonic MPO activity by 83 percent.

Vitamin C or ascorbic acid is water-soluble and absorbed in the small intestinal and colonic cells by ascorbate-specific transporters. The antioxidant properties of ascorbic acid are enhanced by its ability to recycle glutathione and vitamin E. The antioxidant buffering capacity and defense systems within colonic epithelium have been shown to be impaired under clinical and experimental states of inflammation. The novel applications of vitamin C plus 5-ASA, as topical treatment for TNBS colitis in the present study, has demonstrated superiority of this combination therapy versus 5-ASA alone in reducing histological and biomarker (MPO activity) measures of colitis. Results of our studies with vitamin E and vitamin C indicate that inclusion of these antioxidants with mesalamine significantly enhances the pharmacological effectiveness of 5-ASA in topical treatment of TNBS colitis.

In another aspect, disclosed herein is a pharmaceutical composition comprising a therapeutically effective amount of 5-ASA, or a pharmaceutically acceptable salt thereof, and an antioxidant, or a pharmaceutically acceptable salt thereof, and a pharmaceutically acceptable carrier, excipient, or diluent.

As used herein, a “therapeutically effective amount” refers to an amount of a compound that elicits the desired biological or medicinal response in a subject.

As used herein, a “pharmaceutical composition” refers to a mixture of a compound with other chemical components such as diluents, carriers or other excipients. A pharmaceutical composition may facilitate administration of the compound to a subject. Many techniques of administering a compound exist are known in the art, such as, without limitation, orally, intramuscularly, intraocularly, intranasally, parenterally, intravenously and topically. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

Suitable routes of administration may, without limitation, include oral, rectal, or intestinal administration.

Pharmaceutical compositions for use in accordance with the present disclosure thus may be formulated in conventional manner using one or more pharmaceutically acceptable carriers comprising excipients and auxiliaries, which facilitate processing of the active compounds into preparations, which can be used pharmaceutically.

For oral administration, the compounds can be formulated by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

The compounds may also be formulated in rectal compositions such as suppositories or retention enemas, e.g., containing conventional suppository bases such as cocoa butter or other glycerides.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may be used.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### EXAMPLES

### Materials and Methods

### Experimental Animals

Male Sprague Dawley rats weighing 200-250 grams were housed in the animal facility in cages containing contact bedding. Rats were deprived of food but not water for 24 hrs prior to the induction of colitis. Institutional approval for experimental protocols was provided by the research and animal care committees of the Research Services at the Oklahoma City Veterans Administration Medical Center.

### Induction of Colitis

Fasted animals were lightly anesthetized with isoflurane and then subjected to enema administration of TNBS and subsequently study drugs. The experimental protocol is similar to that previously described (20). The tip of polyethylene catheter was advanced transanally 8 cm to the distal colon and a single dose of TNBS (Sigma, St. Louis, Mo.), was instilled intraluminally (15 mg of TNBS dissolved in a 0.6 ml volume of 50% ethanol) to induce colitis. Following the administration of TNBS the animals were maintained in a head-down position for approximately 60 seconds to prevent leakage of the infusate.

Three days after TNBS dosing rats were randomly assigned to receive one of the following treatments by enema in a manner similar to that used for TNBS administration. The TNBS treated animals received one of the following therapies 5-ASA (100 mg/kg) alone or 5-ASA in combination with one of the following: vitamin C (100 mg/kg), phenyl butylnitrone (PBN; 30 mg/kg) and vitamin E (a tocopherol; 50 mg/kg solubilized in DMSO). The doses of selected antioxidants were derived from the literature in which these agents had been used in experimental models of disease or IBD (19, 22, 23). Comparator groups included TNBS dosed rats that received 8 doses of rectal saline and saline treated controls. Rats were treated for 8 days and then sacrificed.

### Assessment of Macroscopic Injury

Upon sacrifice, the distal 10 cm of the colon was removed, opened with a longitudinal incision, and rinsed with phosphate-buffered saline to remove fecal material. Macroscopic assessment of colitis was carried out by an independent observer who was unaware of the treatment groups. The criteria and scale of grading macroscopic injury are listed in Table 1.

Inflammation was present if the mucosa was erythematous. Ulceration of the mucosa was defined as a distinct break or interruption of the mucosa. Once macroscopic damage was assessed full thickness colonic tissue samples were taken from inflamed areas and processed for histology.

TABLE 1

| Macroscopic injury | Score |
|---|---|
| Mo damage | 0 |
| Hyperemia, no ulcers | 1 |
| Linear ulcer with no significant inflammation | 2 |
| Linear ulcer with inflammation at one site | 3 |
| Two or more sites of ulceration and inflammation | 4 |
| Two or more major sites of ulceration and inflammation or | 5 |
| One site of ulceration/inflammation up to 1 cm along the length | |
| of colon | |
| Damage >1 and <2 cm along the length of the colon, | 6 |
| The score is increased by 1 for each additional cm of involvement | |

### Assessment of Microscopic Injury

Histological assessment was used to measure colonic injury. Colonic tissue samples taken from the distal colon were immersed in 10% phosphate buffered formalin and subsequently embedded in paraffin. Sections of 3 micron thickness were stained with hematoxylin and eosin. The slides were then evaluated by a pathologist for epithelial damage, architectural changes, polymorphonuclear leukocyte (PMN) infiltration, mononuclear cell infiltration, and ulceration. The microscopic features of colitis were graded on a 0-3 scale with 0 being normal and 3 representing severe or most intense abnormality. Table 2 depicts the criteria used for assessment of microscopic injury. In addition to scoring individual features of colitis, an aggregate score of colitis for each rat was tabulated by adding together individual parameter scores, thus, providing a global assessment of colitis.

TABLE 2
Criteria for assessment of Microscopic injury

| | |
|---|---|
| Epithelial damage | 0 = Normal |
| | 1 = Focal mucosal injury |
| | 2 = moderate mucosal injury |
| | 3 = Extensive mucosal injury |
| Architectural damage | 0 = Normal |
| | 1 = Mildly disturbed |
| | 2 = Moderately disturbed |
| | 3 = Severely disturbed |
| Mononuclear infiltration | 0 = Normal |
| | 1 = Mild increase |
| | 2 = Moderate increase |
| | 3 = Severe increase |
| PMN infiltration | 0 = Normal |
| | 1 = present in surface epithelium |
| | 2 = Cryptitis |
| | 3 = Crypt abscess |
| Ulceration | 0 = None |
| | 1 = 1%-33% ulcerated |
| | 2 = 34%-66% ulcerated |
| | 3 = ≧67% ulcerated |

### Assessment of Colonic Inflammation by MPO Assay

MPO activity was measured in colonic tissue samples by colorimetric assay. Whole-thickness tissues from the distal colon were weighed (100 mg) and immediately snap-frozen in liquid nitrogen for storage at −80° C. The tissues were then removed from storage and allowed to thaw on ice. Once thawed, 1 ml of hexadecyltrimethylammonium bromide (HTAB; Sigma, St. Louis, Mo.) containing 50 mM KH₂PO₄ (Sigma) and 0.1 M Na₂HPO₄ (Sigma) was added, homogenized and the resultant suspension was then centrifuged at 12,000 g for 10 min at 4° C. The supernatant was collected for measurement of MPO activity.

Horseradish peroxidase (Sigma) was used as a standard; stock solution of 0.5 mg/ml. Tetramethylbenzidine (TMB; Sigma) was used as the substrate for carrying out the reaction. At the time of assay, 25 μl of standard and sample were added to appropriately labeled tubes. TMB was added at a volume of 250 μl to initiate the reaction and 0.1 M H₂SO₄ (250 μl) was added after 10 min to terminate the reaction. The absorbance changes were read at 450 nm and recorded. Results are expressed as nanograms per gram of tissue. MPO activity was used as an indirect measure of the severity of colonic inflammation reflected by PMN leukocyte infiltration.

### Statistical Analysis

All values in the figures and text are expressed as means±standard error of the mean (SEM). The statistical significance of any difference among groups was analyzed using Student's two-tailed t test for equal and unequal variance observations. P values of <0.05 were considered to be statistically significant.

### Results:

### Effects of Antioxidant Plus 5-ASA Therapy on Macroscopic Colitis

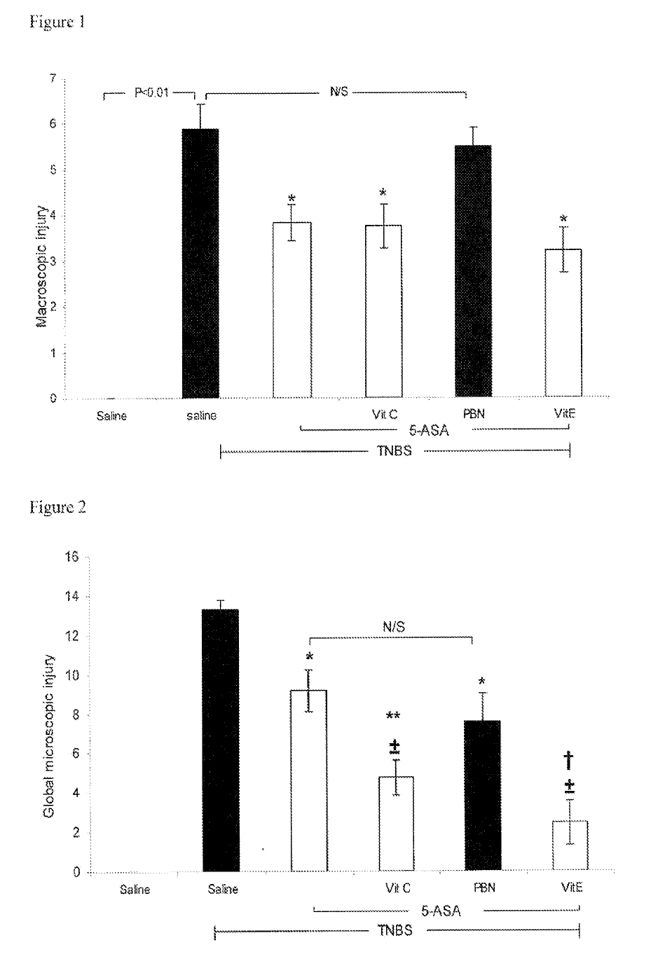

Visual evidence of colitis 11 days after TNBS administration was scored 5.9±0.6 (FIG. 1). Monotherapy with 5-ASA alone resulted in significant reduction in TNBS induced macroscopic injury, 3.8±0.4 (P<0.01). Addition of Vitamin C and Vitamin E to 5-ASA resulted in significant reduction in TNBS-induced macroscopic injury to values of 3.8±0.5 and 3.2±0.5, (P<0.02, and P<0.003), respectively. These results, however, were not different from those achieved with 5-ASA alone. Combination of PBN with 5-ASA, did not resulted in significant reduction in TNBS induced colitis.

### Effects of Antioxidant Plus 5-ASA Therapy on Microscopic Colitis

Global assessments of microscopic colitis induced by TNBS (score 13.3±0.5) indicated that 5-ASA alone significantly reduced colonic injury by 31% (9.1±1.0; P<0.01) (FIG. 2). Combination therapy with either vitamin C plus 5-ASA or vitamin E plus 5-ASA caused further significant reduction in TNBS colitis and resulted in injury scores of 4.7±0.9 (−65%) and 2.4±1.0 (−82%), respectively. Each of these values was not only significantly less than that observed with TNBS (P<0.0001) but they were also significantly below scores observed with 5-ASA as monotherapy (P<0.01 and P<0.004), respectively. Enema therapy with PBN plus 5-ASA resulted in a colitis injury score of 7.5±1.4 which was significantly less than TNBS (P<0.01) but was not different from results with 5-ASA alone (9.1±1.0; P>0.1).

### Effects of Luminal Antioxidant Therapies Plus Mesalamine on Individual Measures of Microscopic Colitis.

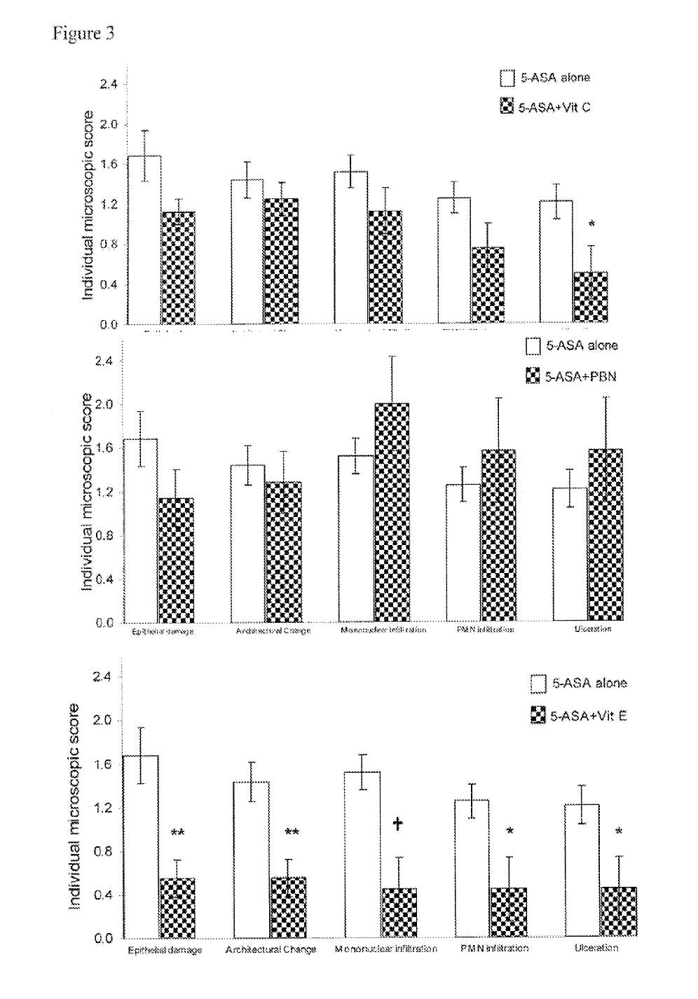

Subset analysis of the separate histological indices of colitis for each antioxidant plus 5-ASA were compared to responses to 5-ASA alone in the treatment of TNBS colitis (FIG. 3). FIG. 3A indicates that combination therapy with vitamin C plus 5-ASA caused numerical reductions in 5 of the 6 measures of colitis but these values did not reach statistical significance. Mucosal ulceration, however, was significantly inhibited by vitamin C plus 5-ASA. The combination of PBN plus 5-ASA caused a variable response in the histological features of colitis and none of these was different from monotherapy with 5-ASA (FIG. 3B). In contrast topical therapy with vitamin E plus 5-ASA caused significant decrease in each histological feature of colitis when compared to 5-ASA alone (FIG. 3C).

Representative photomicrographs of distal colonic specimens from rats subjected to the different treatment protocols were obtained. TNBS colitis, illustrated by the histological specimen obtained 11 days after induction of colitis, was characterized by mucosal ulceration, intense inflammatory cell response in the lamina propria, cystic glandular dilatation and the presence of inflammatory cells within the glandular lumen. In addition, there was a notable lack of goblet cells. The effects of monotherapy with topical 5-ASA which was administered for 8 days and begun 3 days after initiation of TNBS colitis were observed in a photomicrograph. This photomicrograph indicates an area of mucosal ulceration, mild inflammation in the submucosa, cystic glandular dilatation and restoration of goblet cells. Combination therapy with luminally delivered vitamin C plus 5-ASA revealed an absence of mucosal ulceration, the presence of goblet cells, cystic glandular dilation and evidence of glandular branching. Mild inflammation was present in the lamina propria. PBN plus 5-ASA treatment of TNBS colitis was associated with focal mucosal ulceration, marked cystic glandular dilatation containing mucin-like material and moderate inflammation. Topical therapy with vitamin E plus 5-ASA for TNBS colitis resulted in mucosal healing with an absence of mucosal ulceration, replenishment of goblet cells, cystic glandular dilatation with mucin-like material within the lumen, branching within the glandular crypts and minimal inflammation. These histological samples reveal a spectrum of disease activity in response to luminal topical therapies for TNBS colitis. Monotherapy with 5-ASA and combination therapies with specific antioxidants plus 5-ASA showed varying degrees of mucosal repair and reduction in inflammation. In general, the microscopic features of colitis illustrated in these photomicrographs correlate with quantitative measures of colitis described in FIGS. 2 and 3.

### Effects of Antioxidant Plus 5-ASA Therapy on Mucosal Inflammation

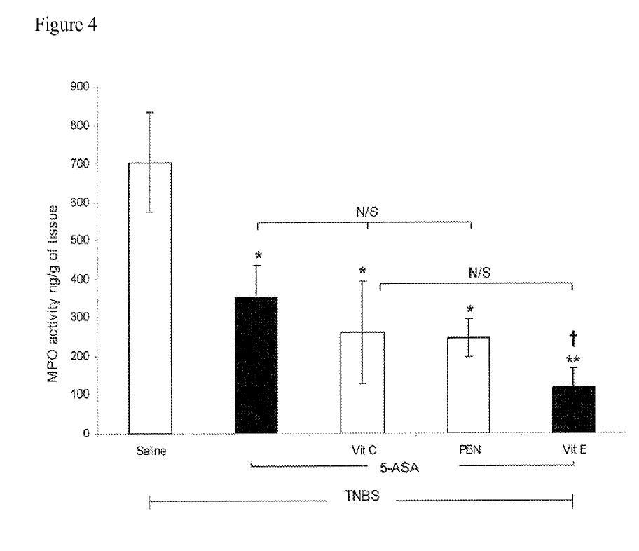

MPO activity in the distal colon (FIG. 4) was decreased significantly in response to monotherapy with 5-ASA alone (354±81 ng/gm; P<0.03) when compared to TNBS-induced colitis (703+130 ng/gm). Furthermore, combination therapy with Vitamin E plus 5-ASA caused reduction in the MPO activity (118±51 ng/gm) that was significantly less than both TNBS and TNBS plus 5-ASA (P<0.002 and P<0.04, respectively). In contrast, luminal therapies vitamin C and PBN plus 5-ASA reduced MPO activity significantly in TNBS colitis (P<0.05) but results were not different from those observed with 5-ASA alone (P>0.1).

What is claimed is:

1. A composition comprising 5-aminosalicylic acid (5-ASA), or a pharmaceutically acceptable salt thereof and an antioxidant, or a pharmaceutically acceptable salt thereof, wherein the antioxidant is not N-acetylcysteine.

2. The composition of claim 1, wherein the antioxidant is selected from the group consisting of vitamin C (ascorbic acid), vitamin E (α-tocopherol), and phenyl butylnitrone.

3. The composition of claim 1, wherein the antioxidant is a mixture of two or more antioxidants.

4. The composition of claim 1, further comprising a pharmaceutically acceptable carrier or excipient.

5. The composition of claim 1, wherein the 5-ASA, or a pharmaceutically acceptable salt thereof, and the antioxidant, or a pharmaceutically acceptable salt thereof, are together disposed in a liquid formulation.

6. The composition of claim 5, wherein the liquid is an aqueous solution.

7. The composition of claim 5, wherein the liquid is an emulsion.

8. The composition of claim 1, wherein the 5-ASA, or a pharmaceutically acceptable salt thereof, and the antioxidant, or a pharmaceutically acceptable salt thereof, are together disposed in a solid formulation.

9. A method of treating an inflammatory bowel disease in a subject, comprising administering to the subject a therapeutically-effective amount of a composition comprising 5-ASA, or a pharmaceutically-acceptable salt thereof, and an antioxidant, or a pharmaceutically acceptable salt thereof.

10. The method of claim 9, wherein the composition is administered to the subject by rectal delivery or oral delivery to a colon or small intestine of the subject.

11. The method of claim 9, wherein the 5-ASA, or a pharmaceutically-acceptable salt thereof, and the antioxidant, or a pharmaceutically-acceptable salt thereof, are together disposed in a pharmaceutically acceptable carrier.

12. The method of claim 9, wherein the 5-ASA, or a pharmaceutically-acceptable salt thereof, and the antioxidant, or a pharmaceutically-acceptable salt thereof, are administered separately.

13. The method of claim 9, wherein macroscopic injury to colonic mucosa of the subject is reduced.

14. The method of claim 9, wherein microscopic injury to colonic mucosa of the subject is reduced.

15. The method of claim 9, wherein inflammation in colonic mucosa of the subject is reduced.

16. The method of claim 9, wherein the subject is a human.

17. The method of claim 9, wherein the antioxidant is selected from the group consisting of vitamin C (ascorbic acid), vitamin E (α-tocopherol), and phenyl butylnitrone.

18. A method of reducing cytokine gene expression in colonic tissue of a subject comprising administering to the subject a therapeutically-effective amount of a composition comprising 5-ASA, or a pharmaceutically-acceptable salt thereof, and an antioxidant, or a pharmaceutically acceptable salt thereof.

19. The method of claim 18, wherein the cytokine is selected from the group consisting of IL 1a, IL 1b, IL-4, IL-6, and TNFα.

20. The method of claim 17, wherein the 5-ASA, or a pharmaceutically-acceptable salt thereof, and the antioxidant, or a pharmaceutically acceptable salt thereof, are together disposed in a pharmaceutically acceptable carrier.

21. The method of claim 18, wherein the 5-ASA, or a pharmaceutically-acceptable salt thereof, and the antioxidant, or a pharmaceutically-acceptable salt thereof, are administered separately.

22. The method of claim 18, wherein macroscopic injury or inflammation to colonic mucosa of the subject is reduced.

23. The method of claim 18, wherein microscopic injury to colonic mucosa of the subject is reduced.

24. The method of claim 18, wherein the subject is a human.

25. The method of claim 18, wherein the antioxidant is selected from the group consisting of vitamin C (ascorbic acid), vitamin E (α-tocopherol), and phenyl butylnitrone.

26. A method of reducing myeloperoxide activity in colonic tissue of a subject comprising administering to the subject a therapeutically-effective amount of a composition comprising 5-ASA, or a pharmaceutically-acceptable salt thereof, and an antioxidant, or a pharmaceutically acceptable salt thereof.

27. The method of claim 26, wherein the 5-ASA, or a pharmaceutically-acceptable salt thereof, and the antioxidant, or a pharmaceutically-acceptable salt thereof, are together disposed in a pharmaceutically acceptable carrier.

28. The method of claim 26, wherein the 5-ASA, or a pharmaceutically-acceptable salt thereof, and the antioxidant, or a pharmaceutically-acceptable salt thereof, are administered separately.

29. The method of claim 26, wherein macroscopic injury or inflammation to colonic mucosa of the subject is reduced.

30. The method of claim 26, wherein microscopic injury to colonic mucosa of the subject is reduced.

31. The method of claim 26, wherein the subject is a human.

32. The method of claim 26, wherein the antioxidant is selected from the group consisting of vitamin C (ascorbic acid), vitamin E (α-tocopherol), and phenyl butylnitrone.
